(19) United States
(12) Patent Application Publication
(10) Pub. No.: US 2025/0339469 A1
(43) Pub. Date: Nov. 6, 2025

(54) METHODS FOR ISOLATING CELLS FROM A TISSUE SAMPLE.

(71) Applicant: CartiRegen B.V., Driebergen-Rijsenburg (NL)

(72) Inventors: Clayton Ellis WILSON, Driebergen-Rijsenburg (NL); Willem Cornelis DE JONG, Driebergen-Rijsenburg (NL)

(21) Appl. No.: 18/864,392

(22) PCT Filed: May 10, 2023
(86) PCT No.: PCT/NL2023/050253

§ 371 Date: Nov. 8, 2024

(30) Foreign Application Priority Data
May 10, 2022 (NL) 2031814

(51) Int. Cl. A61K 35/32; A23L 13/72; C12N 5/077

(52) U.S. Cl. CPC A61K 35/32; A23L 13/72; C12N 5/0655; C12N 2509/00

(57) ABSTRACT

The invention provides inter alia a method for enzymatic digestion of an extracellular matrix protein in a tissue sample, comprising the steps of:—providing an aqueous medium that comprises (i) a matrix metalloproteinase and (ii) a cation that is Ca²⁺ at a concentration of at least 2 mmol/L;—contacting said aqueous medium with a tissue sample that comprises an extracellular matrix protein under conditions that allow for enzymatic digestion of said extracellular matrix protein; wherein said tissue sample is a cartilage tissue sample.

### FIELD OF THE INVENTION

The invention relates to methods for enzymatic digestion of extracellular matrix (ECM) proteins. The in vitro or ex vivo methods foreseen herein allow for tissue dissociation and release of cells from said tissue, which cells may subsequently be isolated and formulated for use in medical treatment methods. The in vitro or ex vivo enzymatic digestion methods of the invention provide for improved yields of isolated cells per tissue mass unit. The invention also relates to aqueous media comprising matrix metalloproteinases, which can be used in applications where breakdown of extracellular matrix is beneficial, such as in the treatment of diseases associated with pathological accumulation of extracellular matrix, preferably pathological accumulation of extracellular matrix in, or on, skin or in body orifices and their associated cavities. Other applications that benefit from extracellular matrix degradation using aqueous media as disclosed herein are for instance meat tenderizing and fish deboning.

### BACKGROUND TO THE INVENTION

In the last decades, there has been a rise in the use of cell-based medicine. Cell-based medicine covers different fields such as cell transplantation, regenerative medicine, and tissue engineering. Examples of cell-based medicine are for instance cardiac cell patches to repair heart lesions, renal organoids to restore kidney function, pancreatic organoids to restore pancreas function, intralesional injection of stromal cells from fat or bone marrow to initiate or to enable a repair response, etcetera. The commonality of these cell-based therapeutic strategies is that they require the presence of sufficient quantities of tissue-isolated cells. Frequently, autologous cells are used in cell-based medical treatment methods, whereby cells are harvested from a patient's own tissue to avoid immunological rejection. Isolation of cells can be done in a number of ways, but may involve disruption of the extracellular matrix of a harvested tissue sample by enzymatic digestion with matrix metalloproteinases, such as collagenase, to dissociate tissue and release the cells contained in the extracellular matrix.

One example of a tissue that is of interest in relation to cell-based medicine is cartilage tissue. Cartilage tissue comprises chondrocytes that are embedded in an extracellular matrix. In healthy cartilage tissue, chondrocytes produce and maintain components of the extracellular matrix, such as collagen fibrils, proteoglycans, glycosaminoglycans and elastin. Cartilage tissue may be damaged by for example chondrogenesis disorders, arthritis, articular cartilage trauma and meniscus injury. Cartilage tissue may be repaired by transplantation of autologous chondrocytes into the cartilage tissue lesion. For autologous transplantation of chondrocytes, chondrocytes need to be isolated first from a cartilage tissue sample of a subject, for instance by degrading or breaking down the extracellular matrix in said tissue sample by enzymatic digestion in order to release said chondrocytes. Alternatively, smaller cartilage lesions may be treated with a solution of ECM-degrading enzymes, which can result in increased permeability of the ECM and, subsequently, increased migratory potential of resident perilesional chondrocytes.

Matrix metalloproteinases, which may also be referred to as matrix metalloproteases, are a class of endoproteases that break down extracellular matrix proteins by hydrolysis of peptide bonds, including several types of collagens, gelatins, elastins, fibronectins and lamellins. An especially prominent matrix metalloproteinase are collagenases. Collagenases are known to be produced by animals, prokaryotes, fungi, and plants (Kim et al., Biochim Biophys Acta. 2007; 1770(12):1627-35) and can break down several types of collagen and gelatin.

Philominatan et al., FEBS J. 2009; 276(13):3589-3601 describe the effect of Ca²⁺ on conformational changes of a Clostridial collagenase, but Ca²⁺ concentrations were substantially lower than 2 mmol/L.

Ohbayashi et al., Appl Environ Microbiol. 2012; 78(16):5839-5844 describe that Ca²⁺ has an effect on the structural stability and thermostability of Clostridial ColH, a class-II collagenase. However, no effect of Ca²⁺ on ECM digestion efficacy was tested.

WO 2008/026928 A1 describes cartilage repair implants and mentions in relation to chondrocyte isolation that, prior to subjecting the tissue sample to a digestion enzyme, the tissue sample is subjected to a treatment to increase extracellular matrix permeability such as contacting the tissue sample with an acid, base, dimethyl sulfoxide (DMSO), cathepsin, glycerol or a cation including Na⁺, K⁺, NH₄⁺, Pb²⁺, Mg²⁺, Zn²⁺, Fe²⁺, Cd²⁺ and Cu²⁺. These cations may for instance be introduced in the form of their chloride salts in a concentration between 10 mM and 2M. It is indicated that, after permeability is increased, the tissue sample is washed before subjecting it to a digestion enzyme.

Although it is known that collagenases contain zinc and require calcium to be active (Khokha and Denhardt, Invasion Metastasis. 1989; 9(6):391-405), the effect of supraphysiological concentrations of Ca²⁺ (i.e. concentrations of at least 2 mmol/L) in the aqueous digestion medium on the efficacy of collagenase-based ECM digestion has hitherto not been investigated.

There is a need in the art for further cell isolation methods, especially methods that allow for cells to be isolated in high yields from tissue samples that contain an ECM. There is also a need for aqueous media comprising digestion enzymes that can be used in therapeutic and non-therapeutic methods wherein breakdown or degradation of ECM is advantageous.

### SUMMARY OF THE INVENTION

The inventors have unexpectedly established that, when incubating a matrix metalloproteinase with a tissue sample in an aqueous medium to break down ECM and release cells from said tissue sample, a supraphysiological concentration of calcium ions of at least 2 mmol/L allows for improved isolated cell yield per tissue sample.

More specifically, the inventors surprisingly identified that the simultaneous exposure of a cartilage tissue sample to both a matrix metalloproteinase and a supraphysiological concentration of calcium ions of at least 2 mmol/L in an aqueous medium allows for improved isolated cell yield per cartilage tissue sample.

Therefore, the invention provides a method for enzymatic digestion of an extracellular matrix protein in a tissue sample, comprising the steps of:-providing an aqueous medium that comprises (i) a matrix metalloproteinase and (ii) a cation that is Ca²⁺ at a concentration of at least 2 mmol/L;-contacting said aqueous medium with a tissue sample that comprises an extracellular matrix protein under conditions that allow for enzymatic digestion of said extracellular matrix protein; wherein said tissue sample is a cartilage tissue sample.

In a preferred embodiment of said method for enzymatic digestion, said aqueous medium is prepared by a method comprising the steps of:—providing an aqueous medium;—dissolving a calcium salt in said aqueous medium, wherein the final concentration of Ca²⁺ in said aqueous medium is at least 2 mmol/L; and—mixing, prior or after said step of dissolving, a matrix metalloproteinase with said aqueous medium.

In another preferred embodiment of said method for enzymatic digestion, said step of contacting said aqueous medium with said tissue sample provides for at least partial dissociation of said tissue sample thereby releasing a cell from said tissue sample in said aqueous medium.

In another preferred embodiment of said method for enzymatic digestion, said method is a method for isolating a cell, either with or without its pericellular matrix, from a tissue sample.

In another preferred embodiment of said method for enzymatic digestion, said method further comprises a step of:—isolating a cell from said aqueous medium after enzymatic digestion, preferably by passing a digestate through a cell strainer.

In another preferred embodiment of said method for enzymatic digestion, said matrix metalloproteinase is selected from the group consisting of a collagenase, a gelatinase, a stromelysin, a matrilysin, a metalloelastase, an enamelysin, an endometase and an epilysin.

In another preferred embodiment of said method for enzymatic digestion, said matrix metalloproteinase is a collagenase.

In another preferred embodiment of said method for enzymatic digestion, said matrix metalloproteinase is exclusively restricted to only one matrix metalloproteinase selected from the group consisting of a collagenase, a gelatinase, a stromelysin, a matrilysin, a metalloelastase, an enamelysin, an endometase and an epilysin.

In another preferred embodiment of said method for enzymatic digestion, said matrix metalloproteinase is exclusively restricted to collagenase.

In another preferred embodiment of said method for enzymatic digestion, a collagenase is the only protease present in the aqueous medium.

In another preferred embodiment of said method for enzymatic digestion, said aqueous medium comprises a cation that is Ca²⁺ at a supraphysiological concentration of above 10 mmol/L, for instance above 15 mmol/L.

In another preferred embodiment of said method for enzymatic digestion, said aqueous medium comprises dissolved CaCl₂.

In another preferred embodiment of said method for enzymatic digestion, said method does not comprise a separate incubation step of the tissue sample in a solution for increasing the permeability of the extracellular matrix, such as a cation solution, and/or a separate washing step of the tissue sample prior to incubating the tissue sample in the aqueous medium of the present invention. It was established that such a separate incubation step of the tissue sample in a solution for increasing the permeability of the extracellular matrix, such as a cation solution, and/or a separate washing step prior to incubating the tissue sample in the aqueous medium of the present invention, is not required in order to achieve beneficial cell yields.

In another preferred embodiment of said method for enzymatic digestion, said tissue sample is not pancreas tissue and/or muscle tissue, preferably said sample is not pancreas tissue comprising islet cells and/or muscle tissue comprising smooth muscle cells.

In another preferred embodiment of said method for enzymatic digestion, said tissue sample is a harvested or biopsied tissue sample, optionally a harvested or biopsied tissue sample that is minced prior to said step of contacting said aqueous medium with said tissue sample.

In another preferred embodiment of said method for enzymatic digestion, said cell is a chondrocyte, either with or without its pericellular matrix, and said tissue sample is a cartilage tissue sample, preferably an articular cartilage tissue sample. Preferably, the cartilage tissue sample, such as the articular cartilage tissue sample, is an animal tissue sample, e.g., a human tissue sample, an equine tissue sample or a bovine tissue sample.

In another aspect, the invention provides an aqueous medium comprising (i) a matrix metalloproteinase and (ii) a cation that is Ca²⁺ at a concentration of at least 2 mmol/L.

In a preferred embodiment of said aqueous medium, said matrix metalloproteinase is a collagenase.

In another preferred embodiment of said aqueous medium, said matrix metalloproteinase is exclusively restricted to only one matrix metalloproteinase selected from the group consisting of a collagenase, a gelatinase, a stromelysin, a matrilysin, a metalloelastase, an enamelysin, an endometase and an epilysin.

In a preferred embodiment of said aqueous medium, said matrix metalloproteinase is exclusively restricted to a collagenase.

In another preferred embodiment of said aqueous medium, collagenase is the only protease present in the aqueous medium.

In another preferred embodiment of said aqueous medium, said aqueous medium comprises a cation that is Ca²⁺ at a supraphysiological concentration of above 10 mmol/L, for instance above 15 mmol/L.

In another preferred embodiment of said aqueous medium, said aqueous medium does not comprise pancreas tissue and/or muscle tissue, preferably said aqueous medium does not comprise pancreas tissue comprising islet cells and/or muscle tissue comprising smooth muscle cells.

In another preferred embodiment of said aqueous medium, said aqueous medium further comprises an cartilage tissue sample.

In another preferred embodiment of said aqueous medium, said aqueous medium further comprises an articular cartilage tissue sample, a chondrocyte and/or a chondron.

In another aspect, the invention provides a container comprising an aqueous medium of the invention.

In another aspect, the invention provides a kit comprising a container of the invention.

In another aspect, the invention provides a use of an aqueous medium of the invention in extracellular matrix degradation; wherein said extracellular matrix comprises an extracellular matrix protein, preferably a collagen.

In another aspect, the invention provides a use of an aqueous medium of the invention in enzymatic digestion of an extracellular matrix protein, preferably a collagen.

In another aspect, the invention provides an aqueous medium of the invention for use as a medicament.

In another preferred embodiment of said aqueous medium for use as a medicament, said aqueous medium is for use in a method of treating a cartilage tissue injury.

In another preferred embodiment of said aqueous medium for use as a medicament, said aqueous medium is for use in a method for treating a disorder associated with pathological accumulation of extracellular matrix, preferably pathological accumulation of extracellular matrix in or on skin or in a body orifice or its associated cavity.

In another preferred embodiment of said aqueous medium for use as a medicament, said aqueous medium is for use in a method of treating a wound, burn, keloid disease or retained placenta, or is for use in wound debridement. In the same manner, the invention provides a method for treating a disorder associated with pathological accumulation of extracellular matrix, preferably pathological accumulation of extracellular matrix in or on skin or in a body orifice or its associated cavity, said method comprising the step of:—administering a therapeutically effective amount of an aqueous medium of the invention to a subject in need thereof.

In a preferred embodiment of said method for treating, said method is a method for treating a wound, burn, keloid disease or retained placenta, or is for use in wound debridement.

In the same manner, the invention provides a use of an aqueous medium of the invention in the manufacture of a medicament for treating a disorder associated with pathological accumulation of extracellular matrix, preferably pathological accumulation of extracellular matrix in or on skin or in a body orifice or its associated cavity.

In a preferred embodiment of said use of an aqueous medium, said method is a method for treating a wound, burn, keloid disease or retained placenta, or is for use in wound debridement.

In another aspect, the invention provides a method for tenderizing a meat product, comprising the steps of:—providing an aqueous medium that comprises (i) a matrix metalloproteinase and (ii) a cation that is Ca²⁺ at a concentration of at least 2 mmol/L;—contacting said aqueous medium with a meat product that comprises an extracellular matrix protein under conditions that allow for enzymatic digestion of said extracellular matrix protein.

In another aspect, the invention provides a method for deboning a fish product, comprising the steps of:—providing an aqueous medium that comprises (i) a matrix metalloproteinase and (ii) a cation that is Ca²⁺ at a concentration of at least 2 mmol/L;-contacting said aqueous medium with a fish product that comprises an extracellular matrix protein under conditions that allow for enzymatic digestion of said extracellular matrix protein.

The invention also provides a method for enzymatic digestion of an extracellular matrix protein in a tissue sample, comprising the steps of:—providing an aqueous medium that comprises (i) a matrix metalloproteinase and (ii) a cation, preferably a monovalent or divalent atomic cation; wherein said cation is at a supraphysiological concentration;—contacting said aqueous medium with a tissue sample that comprises an extracellular matrix protein under conditions that allow for enzymatic digestion of said extracellular matrix protein.

Without being bound by theory, the activity of a matrix metalloproteinase, such as collagenase, may be enhanced in swollen tissue samples. The swelling of a tissue sample, such as cartilage tissue that has many negatively charged components, e.g., negatively charged macromolecules, may be facilitated by applying, simultaneously with a matrix metalloproteinase, a supraphysiological concentration of calcium ions which results in an increase of osmolarity and influx of water into the tissue sample.

The invention also provides a method for treating a subject with a cartilage tissue injury, such as a meniscus injury (e.g., torn meniscus), said method comprising the steps of:—harvesting or biopsying a cartilage tissue sample, such as meniscus tissue sample, from a subject with a cartilage tissue injury, such as a meniscus injury;—optionally mincing said tissue sample;—performing a method for enzymatic digestion as disclosed herein; isolating chondrocytes and/or chondrons from said aqueous medium after enzymatic digestion; re-seeding or transplanting said isolated (autologous) chondrocytes and/or chondrons on or in said cartilage tissue of said subject that is injured such as in or on the cartilage tissue lesion of said injured cartilage tissue.

In the same manner, the invention provides an aqueous medium of the invention, or a population of chondrocytes and/or chondrons isolated from said aqueous medium, for use in a method of treating a subject with a cartilage tissue injury, said method comprising the step of:—applying, re-seeding or transplanting (i) an aqueous medium as disclosed herein comprising chondrocytes and/or chondrons (preferably wherein said aqueous medium is a digestate as disclosed herein that has been passed through a strainer) or (ii) a population of isolated (autologous) chondrocytes and/or chondrons, on or in said cartilage tissue of said subject that is injured such as in or on the cartilage tissue lesion (site) of said injured cartilage tissue; preferably wherein said population of isolated chondrocytes and/or chondrons is obtainable by a method comprising the steps of:—providing (or harvesting or biopsying) a cartilage tissue sample, such as meniscus tissue sample, from a subject with a cartilage tissue injury, such as a meniscus injury (e.g., torn meniscus);—optionally mincing said tissue sample;—performing a method for enzymatic digestion of the invention; isolating chondrocytes and/or chondrons from said aqueous medium after enzymatic digestion.

In another aspect, the invention provides a method for the production of a peptide or protein hydrolysate, comprising the steps of:—providing an aqueous medium of the invention; and—contacting said aqueous medium with a composition comprising a protein under conditions that allow for enzymatic digestion of said protein.

In a preferred embodiment of said method, the peptide is a bioactive peptide such as a collagen peptide and the protein is a collagen.

In another preferred embodiment of said method, the protein hydrolysate is a collagen hydrolysate and the protein is a collagen.

### DESCRIPTION OF THE DRAWINGS

FIG. 1. Yields in million per gram (10⁶/g) of primary chondrocytes isolated with an enzymatic digestion from equine cartilage. The control condition contained a calcium ion concentration of 0.423 mM. The chondrocyte yield in the control situation was 1.9×10⁶ g⁻¹ versus 3.5×10⁶ g⁻¹ when the calcium ion concentration in the digestion medium was 10 mM.

FIG. 2. Yields in million per gram (10⁶/g) of primary chondrocytes isolated with an enzymatic digestion from bovine cartilage. The control condition contained a calcium ion concentration of 0.423 mM. The chondrocyte yield in the control situation was 3.4×10⁶ g⁻¹ versus 4.5×10⁶ g⁻¹ when the calcium ion concentration in the digestion medium was 3 mM and 5.0×10⁶ g⁻¹ when the calcium ion concentration in the digestion medium was 10 mM.

### DETAILED DESCRIPTION OF THE INVENTION

### Definitions

The term ‘a’ or ‘an’, as used herein, for instance in relation to matrix metalloproteinases, includes reference to one or more than one matrix metalloproteinase.

The term “enzymatic digestion”, as used herein, includes reference to the chemical conversion or subdivision of macromolecules by means of enzymatic activity. Preferably, the term relates to the proteolytical cleavage of peptide bonds in a protein by an enzyme such as a matrix metalloproteinase. Preferably, enzymatic digestion as disclosed herein involves hydrolysis of a protein.

The term “extracellular matrix”, as used herein, includes reference to the network of macromolecules and minerals that provide structural support and/or protection to the cells in its direct or indirect vicinity, and that interacts with cells via cell surface receptors. The extracellular matrix comprises the interstitial matrix and the basement membrane. Major components of the animal extracellular matrix comprise proteoglycans, non-proteoglycan polysaccharides, extracellular vesicles, and extracellular matrix proteins. The extracellular matrix may embed a variety of cell types depending on the tissue type.

The term “extracellular matrix protein”, as used herein, includes reference to a protein that is part of the extracellular matrix. In animals, extracellular matrix proteins comprise amongst others collagens, elastins, fibronectins, laminins, actins, and integrins. Preferably, the extracellular matrix as disclosed herein is an extracellular matrix of an animal, such as human, tissue sample. Preferably, the extracellular matrix protein as disclosed herein is a collagen.

The term “tissue”, as used herein, includes reference to a cellular organizational level between cells and a complete organ. The term includes reference to cells with their extracellular matrix that together carry out a specific function. Non-limiting examples of tissue include connective, epithelial, muscle and nervous tissue.

The term “tissue sample”, as used herein, includes reference to a tissue that is obtained from a subject for instance by harvesting or as a biopsy. The tissue sample can be obtained from an animal subject, for instance a human subject. The tissue sample is generally a tissue part, wherein said part comprises cells with their extracellular matrix. The skilled person understands that a method for enzymatic digestion as disclosed herein can be performed in vitro or ex vivo if cells need to be isolated e.g., for subsequent autologous or allogenic transplantation. Alternatively, aqueous media as disclosed herein can be used for the treatment of diseases that are associated with pathological accumulation of extracellular matrix. The term also includes reference to any other non-animal tissue that contains an extracellular matrix.

The terms “incubating” and “incubation”, as used herein, includes reference to the active or passive facilitation of contact between two or more chemicals, mixtures, solutions, suspensions, emulsions, extractants, crystals, sols, colloids, gels, biological materials, tissues, media, or a combination thereof, for a period of time. No limit to the length of the time period is intended. External conditions, such as temperature, pH and agitation, may be part of the incubation process. Incubation allows reactions, such as enzymatic digestion reactions, to occur. Incubation can take place with or without an incubator vessel. The term “incubation”, as used herein in relation to incubating an aqueous medium as disclosed herein with a tissue or a tissue sample, can be used interchangeably with the phrase “contacting said aqueous medium with a tissue sample under conditions that allow for enzymatic digestion of said extracellular matrix protein”. The skilled person is well aware of appropriate conditions that allow for enzymatic digestion to take place.

The term “aqueous medium”, as used herein, includes reference to a liquid water-based medium, for instance a liquid medium comprising water as a primary constituent. The medium may further comprise salts, anions, cations, carbohydrates, macromolecules, cells, fats, fatty acids, other solvents, any other organic or inorganic component, or a combination thereof. Preferably, the aqueous medium is a liquid aqueous medium such as a solution, suspension or colloid.

The term “cation”, as used herein, includes reference to a positively charged ion. The cation can for instance be a monovalent or divalent atomic ion, such as a monovalent or divalent metal ion. Preferably, the cation is a divalent metal ion such as Ca²⁺. Other cations that may find application in the present invention are Na⁺, K⁺, NH₄⁺, Pb²⁺, Mg²⁺, Mn²⁺, Zn²⁺, Fe²⁺, and Cu²⁺or Co²⁺, Se⁴⁺, or Cr³⁺.

The term “concentration”, as used herein, includes reference to the molar concentration (mol/L) of compounds in an aqueous medium. In the context of a cation in an aqueous medium, concentration refers to the concentration of said cation prior to said step of contacting said aqueous medium with a tissue sample, meat product or fish product as disclosed herein.

The term “matrix metalloproteinase (MMP)”, as used herein, includes reference to a group of endoproteases which hydrolyze extracellular matrix proteins, and whose function is dependent on the presence of at least one metal such as calcium. Matrix metalloproteinases (MMPs) are also referred to as matrix metallopeptidases or matrixins. Preferably, the MMP is a collagenase, a gelatinase, a stromelysin, a matrilysin, a metalloelastase, an enamelysin, an endometase or an epilysin. The group of matrix metalloproteinases includes the gene products of human genes MMP1, MMP2, MMP3, MMP7, MMP8, MMP9, MMP10, MMP11, MMP12, MMP13, MMP14, MMP15, MMP16, MMP17, MMP19, MMP20, MMP21, MMP23A, MMP23B, MMP24, MMP25, MMP26, MMP27 and MMP28, their post-translationally modified and/or active form, and all their animal, plant, fungal, other eukaryotic, bacterial, archaeal and viral homologues, orthologues, paralogues and counterparts. More preferably, the matrix metalloproteinase is a collagenase such as MMP1, MMP2, MMP8, MMP9 or MMP13.

The term “mixing”, as used herein in relation to the mixing of an MMP with an aqueous medium, includes reference to combining the MMP (which may be provided in a solid form for instance in the form of a lyophilized, non-sterile powder, or pre-dissolved in a liquid medium) and said aqueous medium, for instance by adding the MMP, in solid form or dissolved form, to an aqueous medium while gently stirring.

The term “dissociation”, as used herein in relation to dissociation of a tissue sample upon enzymatic digestion, includes reference to the at least partial loss of integrity of said tissue sample caused by enzymatic digestion of ECM proteins in said tissue sample, and release of cells from said tissue sample. Cells released from said tissue sample tend to be no longer contained in said tissue sample, but may still be bound to other cells and/or other components that were previously part of said tissue such as their pericellular matrix. An example of dissociation of a tissue sample is the at least partial dissociation of a cartilage tissue sample upon enzymatic digestion and release of chondrocytes, either with or without their pericellular matrix, from said cartilage tissue sample. The term “cell”, as used herein, includes reference to cells with or without their pericellular matrix. A preferred cell is a chondrocyte or a chondron. A chondron is a chondrocyte with a pericellular matrix. If reference is made to chondrocytes herein, it includes reference to chondrons.

The term “collagenase”, as used herein, includes reference to a genus of enzymes within the group of matrix metalloproteinases (MMP). Collagenases are known to break peptide bonds of, but not exclusively, collagen and pro-collagen. Preferably, collagenases break peptide bonds through hydrolysis. The genus of collagenases comprises amongst others interstitial collagenase, neutrophil collagenase, collagenase 3, collagenase 4, xenopus collagenase, fibroblast-type collagenase, PMN-type collagenase, type IV collagenase, ColH, ColG, AUX-I and AUX-II. A collagenase as disclosed herein may be of animal, plant, fungal, bacterial or archaeal origin, and may be employed in a method of the invention in purified, partially purified or non-purified form. Collagenase for use in the methods disclosed herein may be commercially or non-commercially produced. Potential substrates of collagenases comprise any type of collagen, including type I collagen, type II collagen, type III collagen, type IV collagen and type V collagen, type VI collagen, type IX collagen, type X collagen, type XI collagen, type XIIA collagen, type XIIB collagen, type XIV collagen, type XVI collagen, type XXII collagen and type XXVI collagen. Another non-limiting example of a potential substrate of collagenases is gelatin. Preferably, the collagenase is a microbial, preferably a bacterial, collagenase.

The term “gelatinase”, as used herein, includes reference to a genus of enzymes within the group of matrix metalloproteinases (MMP). Gelatinases can break peptide bonds of, but not exclusively, collagen, pre-collagen and gelatin. The genus of gelatinases comprises amongst others gelatinase A and gelatinase B. Gelatinases may be of animal, plant, fungal, bacterial or archaeal origin, and may be used in purified, partially purified or non-purified form. Gelatinases for use in the methods of the invention may be commercially or non-commercially produced. Non-limiting examples of substrates of gelatinases include type I gelatin, type V gelatin, type I collagen, type III collagen, type IV collagen, type V collagen, type VII collagen, type X collagen, type XI collagen, tenascin, elastin, fibronectin, laminin-5, vitronectin and entactin.

The term “stromelysin”, as used herein, includes reference to a genus of enzymes within the group of matrix metalloproteinase (MMP). Stromelysin can break peptide bonds of, but not exclusively, collagen and pre-collagen. The genus of stromelysins comprises amongst others stromelysin-1, stromelysin-2, stromelysin-3 and stromelysin-4 (also referred to as RASI-1). Stromelysins may be of animal, plant, fungal, bacterial or archaeal origin, and may be used in purified, partially purified or non-purified form. Stromelysins for use in methods of the invention may be commercially or non-commercially produced. Non-limiting examples of substrates of stromelysin include type II collagen, type IV collagen, type IX collagen, type X collagen, type XI collagen, gelatin, laminin, fibronectin, elastin and aggrecan.

The term “matrilysin”, as used herein, includes reference to a genus of enzymes within the group of matrix metalloproteinase (MMP). Matrilysins can break peptide bonds of, but not exclusively, collagen and pre-collagen. The genus of matrilysins comprises amongst others matrilysin 1 (also referred to as PUMP-1 or uterine metalloproteinase) and matrilysin 2 (also referred to as endometase). Matrilysins may be of animal, plant, fungal, bacterial or archaeal origin, and may be used in purified, partially purified or non-purified form. Matrilysins for use in methods of the invention may be commercially or non-commercially produced. Non-limiting examples of substrates of matrilysins include type III collagen, type IV collagen, type V collagen, type IX collagen, type X collagen, type XI collagen, type I gelatin, type II gelatin, type IV gelatin, type V gelatin, fibronectin, proteoglycan, fibrinogen, casein and vitronectin.

The terms “metalloelastase” and the expression “macrophage metalloelastase”, as used herein, includes reference to an enzyme within the group of matrix metalloproteinase. Metalloelastase can break peptide bonds in, but not exclusively, elastin. Metalloelastase may be of animal, plant, fungal, bacterial or archaeal origin, and may be used in purified, partially purified or non-purified form. Metalloelastase for use in methods of the invention may be commercially or non-commercially produced. Non-limiting examples of substrates of metalloelastase include elastin, type IV collagen, type I gelatin, fibronectin, laminin, vitronectin and proteoglycan.

The term “enamelysin”, as used herein, includes reference to an enzyme within the group of matrix metalloproteinases (MMP). Enamelysin, can break peptide bonds in, but not exclusively, enamelin. Enamelysin may be of animal, plant, fungal, bacterial or archaeal origin, and may be used in purified, partially purified or non-purified form. Enamelysin for use in methods of the invention may be commercially or non-commercially produced. Non-limiting examples of substrates of enamelysin include enamelin and amelogenin.

The term “epilysin”, as used herein, includes reference to a protein within the group of matrix metalloproteinases (MMP). Epilysin can break peptide bonds in, but not exclusively, casein. Epilysin may be of animal, plant, fungal, bacterial or archaeal origin, and may be used in purified, partially purified or non-purified form. Epilysin for use in methods of the invention may be commercially or non-commercially produced.

The terms “dissolved” or “dissolving”, as used herein, includes reference to the formation of a solution comprising at least one solvent and at least one solute.

The terms “cartilage tissue” or “cartilaginous tissue”, which can be used interchangeably, includes reference to tissue from cartilage. Cartilage tissue may comprise tissue from the types elastic cartilage, hyaline cartilage and/or fibrocartilage. Preferably, the cartilage tissue is hyaline cartilage tissue. Cartilage tissue may originate from any type of animal cartilage, including but not limited to joints, rib cage, ear, nose, bronchial tubes and intervertebral discs. Cartilage tissue comprises chondrocytes and an extracellular matrix in which chondrocytes are embedded. The biochemical composition of cartilage tissue is dependent on its location (if applicable, its location before harvesting or performing biopsy) and type, but common features include collagen fibers, elastin fibers, aggrecan, glycosaminoglycans and proteoglycans. Types II collagen is the most prominent collagen type in cartilage, and other types of collagen that may be found in cartilage are type IV collagen, type VI collagen, type IX collagen, type X collagen, type XI collagen, type XII collagen, type XIV collagen, type XIV collagen, type XXII collagen and type XXVII.

Cartilage, preferably hyaline cartilage, comprises negatively charged components such as chondroitin-4 sulphate, chondroitin-6 sulphate, heparan sulphate, and keratan sulphate. The architecture and composition of the extracellular matrix of cartilage differs significantly from that of other tissues, such as pancreas tissue and muscle tissue. The extracellular matrix of cartilage contains significantly more collagen type II and glycosaminoglycans (GAGs) than e.g., that of pancreas tissue and/or muscle tissue. Further, the extracellular matrix of cartilage is more dense and more tightly packed than e.g., that of pancreas tissue and/or muscle tissue. The extracellular matrix represents the main functional component of cartilage, whereas cells represent the main functional component in other tissues, such as e.g., pancreas tissue and/or muscle tissue.

The term “heart tissue”, as used herein, includes reference to tissue from the heart. Heart tissue may comprise ventricular cardiomyocytes, atrial cardiomyocytes, smooth muscle cells, pericytes, fibroblasts, mesothelial cells, endothelial cells, adipocytes and/or cardiac pacemaker cells. Heart tissue preferably comprises an extracellular matrix. Notable components of the extracellular matrix of heart tissue can be type I collagen and type III collagen.

The term “liver tissue”, as used herein, includes reference to tissue from the liver. Liver tissue may comprise hepatocytes, Kuppfer cells, hepatic stellate cells and liver sinusoidal endothelial cells. Liver tissue preferably comprises an extracellular matrix. Notable components of the extracellular matrix of liver tissue are generally type I collagen, type III collagen, type IV collagen and type V collagen.

The term “pancreas tissue”, as used herein, includes reference to tissue from the pancreas. Pancreas tissue may comprises alpha cells, beta cells, delta cells, epsilon cells, PP cells (also referred to as gamma cells) and/or pancreatic connective tissue cells. Pancreas tissue preferably comprises an extracellular matrix. Notable components of the extracellular matrix of pancreas tissue are type I collagen, type III collagen, type IV collagen and type V collagen.

The term “thyroid tissue”, as used herein, refers to tissue from the thyroid. Thyroid tissue may comprise follicular cells and/or parafollicular cells. Thyroid tissue preferably comprises an extracellular matrix. Notable components of the extracellular matrix of thyroid tissue are type I collagen and type III collagen.

The term “salivary gland tissue”, as used herein, include reference to tissue from one of the salivary glands. Salivary gland tissue may comprise serous cells, mucous cells, acinar cells, ductal cells and/or myoepithelial cells. Thyroid tissue preferably comprises an extracellular matrix. A notable component of the extracellular matrix of thyroid tissue is type I collagen.

The term “harvested” or “biopsied”, as used herein, includes reference to having obtained a tissue sample from a subject by methods and means generally known in the art.

The term “subject”, as used herein, preferably relates to a mammal such as a human. Other subjects can be non-human primates, domesticated animals such as dogs, cats, sheep, cattle, goats, pigs, horses etc., laboratory animals such as mice, rats, rabbits, guinea pigs, etc. as well as animals in captivity, such as animals of zoos. In a preferred embodiment, the subject is (i) a human, such as a human patient that is suffering, or suspected of suffering, from a disorder associated with pathological accumulation of extracellular matrix, preferably pathological accumulation of extracellular matrix in or on skin or in a body orifice or its associated cavity, e.g., a wound, burn, keloid disease or retained placenta or (ii) a human that is suffering from a cartilage tissue injury as disclosed herein. With pathological accumulation of extracellular matrix is inter alia meant an overgrowth or excessive deposit of ECM that, when left untreated, provides for a disadvantageous health status.

The term “minced” or “mincing”, as used herein, includes reference to the mechanical division of a tissue sample such as a harvested or biopsied tissue sample, for instance by cutting with a scalpel. The person skilled in the art is aware of generally known methods and means for mincing of a tissue sample.

The term “isolating”, as used herein in relation to isolating a cell from a tissue sample, includes reference to the release of a cell from a tissue sample as a result of applying a method for enzymatic digestion of the invention and/or subsequent retrieval of cells from a digestate for instance by passing said digestate through a cell strainer.

The term “digestate”, as used herein, includes reference to the composition that is obtained after a step of contacting an aqueous medium with a tissue sample that comprises an extracellular matrix protein as disclosed herein, i.e. an aqueous composition comprising enzymatically digested ECM protein, at least partially dissociated tissue sample and/or cells that are released from said tissue sample.

The term “chondrocyte”, as used herein, includes reference to cells that produce components of cartilage tissue and/or maintain the cartilaginous extracellular matrix. The term encompasses chondrons, which are chondrocytes with a pericellular matrix.

The expression “articular cartilage tissue”, as used herein, includes reference to a specific type of cartilage tissue that is found on surfaces of bones with synovial joints. Articular cartilage is an example of hyaline cartilage. Articular cartilage tissue may be obtained from any suitable source such as a meniscus.

The term “treatment”, as used herein, includes reference to the application or administration of an active agent or composition comprising an active agent to a subject, with the aim of partially or completely reversing, alleviating and/or inhibiting the progress of, or prevention of, a disease, or symptoms thereof.

The term “therapeutically effective amount”, as used herein, includes reference to an amount, which has a therapeutic effect or is the amount required to produce a therapeutic effect in a subject. For example, a therapeutically effective amount of an aqueous composition of the invention is the amount required to produce a desired therapeutic effect as may be judged by clinical trial results, model animal studies, and/or in vitro studies. The therapeutically effective amount may depend on several factors, including but not limited to, characteristics of the subject (for example height, weight, sex, age and medical history) and the type of disease.

The term “wound”, as used herein, includes reference to any type of skin tissue injury associated with pathological accumulation of extracellular matrix.

The term “burn”, as used herein, includes reference to a skin wound caused by thermal, chemical or radiation exposure or abrasive friction. The injury may be superficial, or may affect deeper layers of the skin and underlying tissue. Preferably, the burn is associated with a pathological accumulation of extracellular matrix.

The term “keloid disease”, as used herein, includes reference to any disease associated with overgrowth of scar tissue, and which may migrate beyond the boundaries of the original skin injury or lesion.

The term “retained placenta”, as used herein, includes reference to a disorder in which the placenta partially or completely remains in the uterus after (vaginal) birth. Generally, a placenta is retained when it is not expelled within 30 minutes after birth. Retained placenta can for instance be treated by intraplacental administration of an aqueous composition of the invention by injection, or by umbilical cord injection of an aqueous composition of the invention.

The term “wound debridement”, as used herein, includes reference to enzymatic wound debridement which is a standard of care therapy for non-healing and necrotic wounds in subjects for which surgical intervention is not an option. Preferably, the subject is a subject with a non-healing or necrotic wound, and optionally also a subject for which surgical intervention is not an option. Collagenase has been described as the enzymatic debridement product of choice in subjects with for instance wounds containing yellow slough and/or or eschar containing bacteria. Preferably, the wound debridement is enzymatic wound debridement.

The term “tenderizing”, as used herein, includes reference to a process whereby a meat product is rendered more chewable or easier to cut. In general, the breakdown or degradation of extracellular matrix proteins such as collagens in the meat product provides for a meat product that is more tender. Methods of meat tenderization comprise mechanical tenderization, enzymatic tenderization, tenderization with acids, salt solutions, sodium bicarbonate and/or heat. Preferably, in methods of the invention, the tenderizing is enzymatic tenderizing.

The term “meat product”, as used herein, includes reference to meat from a slaughtered animal or artificial meat that is suitable for, or can be made suitable for, human consumption. A meat product generally comprises muscle tissue and/or fat tissue. A meat product can be a meat product from pigs, cows, sheep, chicken, turkeys, goats, ducks and/or deer.

The term “deboning”, as used herein, includes reference to the process of separating fish fillet from fish bone. Deboning of a fish product may be done prior to or following preparation for consumption. Preferably, in a method of the invention, the fish product is a beheaded, gutted and/or fin-cutted fish, which may optionally also be tail-cutted.

The term “fish product”, as used herein, includes reference to a product that comprises both flesh (fillet) of fish and bone. Preferably, the fish product is a beheaded, gutted and/or fin-cutted fish, which may optionally also be tail-cutted. The phrase “body orifice or its associated cavity”, as used herein, includes reference to any body opening and its associated extracellular cavity. Examples of body orifices and their associated cavities include are nostrils and nasal cavity, mouth and throat, ear opening and ear canals, anus and intestines such as rectum, urinary meatus and urethra and urinary bladder, vagina and uterus.

The term “supraphysiological concentration”, as used herein, includes reference to the concentration of a cation such as Ca²⁺ in an aqueous medium of the invention, wherein said concentration is higher than the concentration of said cation normally experienced by said tissue of which a tissue is to be enzymatically digested. For instance, for Ca²⁺, physiological concentrations in different tissue types do not exceed 2 mmol/L. For other cations, it is noted that trace element concentration ranges in mammalian serum are 0.0179-0.0358 mmol/L for iron, 0.0122-0.0184 mmol/L for zinc, 0.00897-0.0157 mmol/L for copper, 0.170-0.509 μmol/L for cobalt, 3.28-3.46 μmol/L for manganese, and 0.633-2.79 nmol/L for selenium (Yatoo et al., Veterinary World. 2013; 6(12):963-967). Cation concentrations of these elements in an aqueous medium of the invention that exceed these ranges (e.g., as measured in serum) may be considered supraphysiological.

The term “bioactive peptide”, as used herein, includes reference to peptides that preferably exhibit a beneficial effect to the human or animal body as a result of its bioactive properties. Bioactive peptides are typically derived from food products, such as protein-comprising material from plant and/or animal products, including cereal grain, milk, eggs, soy, fish and meat. Bioactive peptides are typically obtained by the hydrolysis of whole proteins. Beneficial effects of bioactive peptides include, but are not limited to, influencing human or animal metabolism, preventing disease, reduction of chronic disease, antihypertensive effects, antimicrobial effects, antithrombotic effects, immunomodulatory effects and effects on mineral binding function. Bioactive peptides may be produced enzymatically, via food processing, or by microbial fermentation. Preferably, the composition comprising a protein as disclosed herein comprises collagen (i.e. as an enzymatically digestible protein that can be enzymatically digested by a matrix metalloprotease as disclosed herein in collagen peptides). Further, a composition comprising a protein as disclosed herein can be a whole-protein preparation.

The term “protein hydrolysate”, as used herein, includes reference to whole proteins after digestion, rendering them fragments of proteins. Protein hydrolysate may be more easily and rapidly taken up by the human or animal body compared to whole proteins. Protein hydrolysates are also known in relation to cell culture.

The term “whole-protein preparation”, as used herein, includes reference to proteins that have not been fully digested and are preferably also not (yet) partially digested. These proteins may form the basis for the production of bioactive peptides and/or protein hydrolysates.

### Methods of Isolating Cells From Tissue Through Enzymatic Digestion of ECM

Isolation of cells from tissue has been found to be challenging, and does not always yield a desirable amount of cells. This is especially applicable to tissue types in which the presence of an extracellular matrix prevents cells from being released and isolated from the tissue. An example of such a tissue is cartilage tissue, which contains high amounts of glycosaminoglycans, proteoglycans, collagen and elastin fibers. Isolation of cells from tissue is necessary for multiple cell-based medicine applications. If the isolation process is inefficient, larger tissue sample need to be provided in order to generate a sufficient number of isolated cells.

The present invention now provides a method for efficient isolation of cells from a tissue sample through enzymatic digestion of ECM proteins in a tissue sample using an aqueous medium comprising a matrix metalloproteinase and a cation such as Ca²⁺ at a supraphysiological concentration. This enzymatic digestion method may inter alia be used for the isolation of chondrocytes and/or chondrons from cartilage tissue. As the method of the invention specifically relates to the improved digestion of extracellular matrix proteins in the extracellular matrix of a tissue sample by matrix metalloproteinases, it is also suitable for other processes in which extracellular matrix proteins need to be broken down, such as the treatment of wounds, burns, keloid disease or retained placenta, in enzymatic wound debridement, in meat tenderizing or in fish deboning. The advantageous use of matrix metalloproteinases such as collagenases in such methods has previously already been established. For instance, in relation to enzymatic wound debridement, collagenases are the standard-of-care therapy for non-healing and necrotic wounds in subjects for which surgical intervention is not an option.

Preferably, in a method for enzymatic digestion or isolation of cells from tissue of the invention, a first step involves providing a tissue sample from a subject, for instance through harvesting or as a biopsy. Such a subject can be a subject with a cartilage tissue injury, such as a lesion of the articular cartilage. The skilled person is well aware of suitable methods and means for providing a tissue sample. Further, such a step of harvesting of a tissue sample can be intra-operative, i.e. can be part of a method for treating a subject with a cartilage tissue injury, such as an articular-cartilage lesion, as disclosed herein.

The harvested or biopsied tissue is optionally minced prior to enzymatic digestion to increase the tissue's surface area. Mincing can inter alia be performed by cutting the harvested or biopsied tissue sample with any suitable instrument such as a scalpel.

Subsequently, the tissue sample is contacted with an aqueous medium comprising a matrix metalloproteinase and at least one cation such as Ca²⁺ at a supraphysiological concentration (i.e. as compared to the normal physiological concentration of said cation in said tissue or serum) under conditions that allow for enzymatic digestion of the extracellular matrix protein in said tissue sample. After an appropriate incubation time, under appropriate incubation conditions, the resulting digestate can be strained to filter out undigested tissue using, e.g., a cell strainer with a pore size of 100 μm. The strained digestate can be washed with an appropriate solution, such as phosphate-buffered saline, to wash off or wash out the matrix-metalloproteinase-containing medium. From the washed digestate, which will be a suspension of individual and clustered primary cells with varying amounts of remaining extracellular matrix and extracellular matrix debris, a representative sample may be taken to assess the cell concentration of the digestate. The terms “contacting” and “incubating” can be used interchangeably herein.

The physiological concentration of Ca²⁺ may differ between organ and tissue type. For example, in mammals, the physiological concentration of Ca²⁺ in the extracellular environment is 1-2 mmol/L. The Ca²⁺ concentration in human serum, e.g., is 1.1 to 1.3 mmol/L. In human synovial fluid the Ca²⁺ concentration is 0.32 mmol/L. With physiological concentrations it is meant the concentrations under homeostatic conditions in a living, healthy animal such as a human at or in a tissue of interest, preferably the physiological concentration being the extracellular concentration of a cation as disclosed herein under homeostatic conditions in living, healthy animals at or in a tissue of interest. Supraphysiological concentrations are concentrations exceeding the physiological concentrations at or in said tissue of interest. As the Ca²⁺ concentrations in living, healthy animals are not at least 2 mmol/L, it is accepted that supraphysiological Ca²⁺ concentrations are concentrations of at least, or higher than, 2 mmol/L. In embodiments, the aqueous medium preferably comprises at least 2 mmol/L Ca²⁺ or is higher than 2 mmol/L Ca²⁺, more preferably between 2 and 2000 mmol/L, more preferably between 2 and 200 mmol/L, even more preferably between 2 and 50 mmol/L, and most preferably between 3 and 10 mmol/L such as 3, 4, 5, 6, 7, 8, 9 or 10 mmol/L. The aqueous medium may comprise at least 10 mmol/L Ca²⁺. The aqueous medium may comprise higher than 10, 11, 12, 13, 14, 15, 16, 17, 18, 19, 20, 25, 30, 35, 40, 45 and 50 mmol/L Ca²⁺. The aqueous medium may comprise between 15-50, 15-40, 15-30 or 15-20 mmol/L Ca²⁺. The cation such as Ca²⁺ may be added to an aqueous medium in the form of any salt such as CaSO₄, calcium citrate, CaCO₃, CaBr₂, CaF₂, CaI₂, CaCl₂, Ca(NO₃)₂, CaC₂O₄, Ca(H₂PO₄)₂ or CAHPO₄. Most preferably, the cation that is Ca²⁺ is added to the aqueous medium in the form of its CaCl₂ salt.

In a method of the invention, the enzymatic digestion is performed by one or more matrix metalloproteinases (MMPs), which are contained in an aqueous medium as disclosed herein. Examples of MMPs are collagenases, gelatinases, stromelysins, matrilysins, metalloesterases, enamelysin, endometase and epilysin. MMPs, as used in methods of the invention, include MMPs that are animal, plant, fungal and bacterial MMPs, and homologues, orthologues, paralogues and counterparts thereof such as the MEROPS peptidase family M9 enzymes. Examples of MEROPS peptidase family M9 enzymes are BP, ColH and ColG from Clostridium histolyticum, ColH and class II collagenases from Bacillus cereus, the M9A protein and bacterial collagenase V from Vibrio alginolyticus, VMC peptidase from Vibrio mimicus, ColT and class I collagenases from Clostridium tetani, subfamily M9B protein from Microscilla marina, collagenases from Bacillus licheniformis, Bacillus pumilus, Bacillus subtilis, Rhizoctonia solani, Thermoactinomyces sp. E-21 and Clostridium perfringens. Preferably, the MMP as disclosed herein is a collagenase. More than one collagenase type may be used in methods of the invention. More preferably, a collagenase of type II is used. The amount of MMPs in aqueous medium as disclosed herein is preferably between 0.2% w/v and 10% w/v. Preferably, the amount of MMPs in an aqueous medium is about 2% w/v.

An aqueous media as disclosed herein may further comprise buffering agents to maintain a pH that corresponds to a physiological pH such as a pH of about 7. Buffering agents may be present at concentrations between 1 and 100 mM. Examples of suitable buffering agents include both organic and inorganic compounds and salts thereof, such as citrate, succinate, tartrate, fumarate, gluconate, oxalate, lactate, acetate, phosphate, carbonate, bicarbonate and borate buffers. Additionally, the aqueous medium may comprise amino acid-based buffers, urea buffers and buffers such as Tris, MOPS, ACES, PIPES, BES and HEPES.

Other components that may be comprised in an aqueous medium as disclosed herein are DTPA, EDTA, EGTA; reducing agents, such as dithiotreitol, dithioerythritol, 6-mercaptoethanol, glutathione, thioredoxine, cysteine, ascorbic acid, thioglycolate; ions that are necessary for activation of any of the other components in the medium, such as MgCl₂, NaCl, KCl, ZnCl₂, ZnSO₄; organic solvents or lipid/membrane modifying agents such as DMSO; nonionic detergents such as triton X-100; and/or osmoprotectants such as sucrose.

In preferred embodiments, the method of enzymatic digestion of the invention comprises the step of preparing an aqueous medium as disclosed herein, comprising the steps of:—providing an aqueous medium;-dissolving a calcium salt in said aqueous medium, wherein the final concentration of Ca²⁺ in said aqueous medium is at least 2 mmol/L; and
mixing, prior or after said step of dissolving, a matrix metalloproteinase with said aqueous medium. Preparation can be performed by adding together stock components to reach a desired concentration, and by proper and adequate mixing of said components. Mixing may be performed by pipetting, shaking, stirring, vortex shaking or mixing, sonicating and/or passive mixing. Preferably, mixing is done by vortex shaking. Preferably, mixing of the aqueous medium is performed for a duration of between 0.1 and 20 minutes.

In a method of enzymatic digestion of the invention, the tissue sample that comprises an extracellular matrix protein is mixed with an aqueous media as disclosed herein. Mixing may be done by pipetting, shaking, stirring, vortex shaking or mixing, sonicating and/or passive mixing. Preferably, said tissue sample and medium are mixed by vortex shaking.

Incubating, or contacting, an aqueous medium with a tissue sample as described herein means that said tissue sample is at least partially, preferably fully, submerged in said aqueous medium. During incubation, the tissue sample contacts the aqueous medium and thereby its constituents. During incubation, the container comprising the aqueous medium and tissue sample may be shaken. Preferably, incubation takes place under continuous shaking. The temperature during incubation may be regulated. Preferably, the temperature during incubation is constant. More preferably, the temperature during incubation is constant at about 37° C. Incubation may take place for between 1 and 120 minutes. Preferably, incubation takes place for between 5 and 60 minutes. More preferably, incubation takes for approximately 35 minutes.

In embodiments, the method of the invention comprises a step of isolating or separating a cell from said aqueous medium after enzymatic digestion, preferably by passing a digestate through a cell strainer having a pore size that allows cells to pass and blocks larger components. Preferably, the cell strainer has a pore size of approximately 100 μm.

The tissue sample as disclosed herein can be of any size or shape. Preferably, it is a fragment or subcomponent of a tissue that comprises an ECM as found in the animal body. The tissue sample is preferably an animal tissue sample, more preferably the tissue sample is a human tissue sample.

Preferably, the tissue sample as disclosed herein is selected from the group consisting of a sample of cartilage tissue, heart tissue, liver tissue, pancreas tissue, thyroid tissue, salivary gland tissue, bone tissue, skin tissue, kidney tissue, lung tissue, adipose tissue, tendon tissue, bladder tissue, stomach tissue, colon tissue, esophagus tissue, muscle tissue, nervous tissue, connective tissue and epithelial tissue. More preferably, the tissue sample is selected from the group comprising cartilage tissue, heart tissue, liver tissue, pancreas tissue, thyroid tissue, and salivary gland tissue.

Even more preferably, the tissue sample is a cartilage tissue sample. There are three main types of cartilage tissue, namely elastic cartilage, hyaline cartilage and fibrocartilage tissue. Preferably, the cartilage tissue is of the hyaline cartilage tissue. More preferably, the cartilage tissue is articular cartilage, such as meniscus articular cartilage tissue.

The articular cartilage tissue as disclosed herein can be harvested or biopsied from any suitable location in the animal body. Examples of locations in the animal body suitable for cartilage tissue harvesting are for instance metacarpophalangeal joints, metatarsophalangeal joints, interphalangeal joints, intermetatarsal joints, tarsometatarsal joints, intercuneiform joints, cuboideonavicular joints, cuneonavicular joints, calcaneocuboid joints, talocalcaneonavicular joints, talocalcaneal joints, talocrural joints, tibiofibular joints, patellofemoral joints, tibiofemoral joints, hip joints, sternoclavicular joints, acromioclavicular joints, glenohumeral joints, humeroradial joints, humeroulnar joints, proximal radioulnar joints, distal radioulnar joints, radiocarpal joints, intercarpal joints, midcarpal joints, carpometacarpal joints, intermetacarpal joints, interphalangeal joints, atlanto-axial joints, zygapophysial joints, lumbosacral joints, sacrococcygeal joints, costovertebral joints, sternocostal joints, interchondral joints, costocondral joints, pubic symphysis, sacroiliac joints, temporomandibular joints and atlanto-occipital joints. Preferably, cartilage is harvested from metacarpophalangeal joints and/or metatarsophalangeal joints.

Preferably, in a method for treating a subject with a cartilage tissue injury, a cartilage tissue sample is harvested from the same tissue as the tissue that contains the cartilage tissue injury (which may also be referred to as defect or lesion). For instance, if the cartilage tissue injury is a meniscus injury, the tissue sample that is harvested is preferably a meniscus cartilage tissue sample.

The cells contained in a tissue sample that is subjected to a method for enzymatic digestion of the invention are preferably somatic cells such as primary somatic cells. More preferably, said cells are chondrocytes, cardiomyocytes, hepatocytes or pancreatic islet cells. Even more preferably, said cells are chondrocytes, either with or without their pericellular matrix.

In a method for isolating cells of the invention, the yield of isolated cells is preferably determined by counting. Preferably, a Bürker-Türk slide is used for yield determination. For example, after straining and washing of a digestate, the resultant cell suspension is pipetted up and down repeatedly to homogenize the concentration of the cells in the suspension. Then, a small sample is taken from the homogenized cell suspension and preferably mixed with a dye solution such as Trypan Blue. From this diluted suspension, a 10-L sample is taken twice, to fill each of the two counting chambers of a Burker-Türk slide. Cells are counted for each of the two chambers. The mean count is used to calculate the cell concentration in the chamber, which together with the Trypan Blue dilution factor, allows for a calculation of the cell concentration in the original sample. This concentration is assumed to be representative of the homogenized cell suspension.

The enzymatic digestion method of the invention may further be used for other application where ECM breakdown or degradation is beneficial, such as meat tenderizing or fish deboning. Tenderizing makes meat less chewy and more suitable, pleasant or acceptable for human consumption. In meat tenderizing, the components of the extracellular matrix are partially broken down. Collagen is a major component that needs to be broken down in order to make meat more tender. Embodiments disclosed herein in relation to a method for enzymatic digestion of the invention also apply in relation to a method for tenderizing a meat product of the invention.

A method for enzymatic digestion of the invention may also be used for fish deboning. Fish bones are attached to the fillet and cannot easily be removed prior to cooking. In enzymatic fish deboning, the components of the extracellular matrix are partially broken down. Collagen is a major component of fish bones and is one of the components that attaches the fish bone to the fillet. Embodiments disclosed herein in relation to a method for enzymatic digestion of the invention also apply in relation to a method for tenderizing a meat product of the invention.

The invention also provides a method for the production of a peptide or protein hydrolysate. Said method comprises the step of:—providing an aqueous medium as disclosed herein. Said method also comprises the step of-contacting said aqueous medium with a composition comprising a protein under conditions that allow for enzymatic digestion of said protein.

Preferably, said peptide as produced by said method is a bioactive peptide such as a collagen peptide, and said protein as used in said method is collagen.

Preferably, said protein hydrolysate as produced by said method is a collagen hydrolysate, and said protein as used in said method is collagen.

Preferably, said bioactive peptide and/or said collagen hydrolysate consists of between 2 and 100 amino acid residues. More preferably, said bioactive peptide and/or said collagen hydrolysate consists of between 2 and 20 amino acid residues. Preferably, said bioactive peptide and/or said collagen hydrolysate is safe for human and/or animal consumption, in such a way that normal use of the bioactive peptide or collagen hydrolysate by healthy, non-allergic human beings and/or animals does not cause significant disease or illness.

Preferably, said bioactive peptide hydrolysate is beneficial to human and/or animal health. Said bioactive peptide may for example influence human and/or animal metabolism, prevent disease, reduce chronic disease, directly or indirectly cause antihypertensive effects, directly or indirectly cause antimicrobial effects, directly or indirectly cause antithrombotic effects, directly or indirectly cause immunomodulatory effects and/or directly or indirectly cause effects on mineral binding function.

Preferably, said bioactive peptide and/or said collagen hydrolysate is used in the production of a food product for human or animal consumption. Examples of said food product include formula milk, infant nutrition and protein powders.

Preferably, said bioactive peptide and/or said collagen hydrolysate is produced from a food product that comprises one or more protein. Said food products may for example include cereal grain, milk, eggs, soy, fish and meat.

Preferably, said bioactive peptide and/or said collagen hydrolysate is the result of the hydrolysis of said one or more protein.

An aqueous medium as disclosed herein may also find application in therapy. The use of collagenases in the treatment of disorders that are associated with pathological accumulation of extracellular matrix is well established. For instance, administration of collagenases are commonly used in wound debridement. Other indications where collagenases have been employed, and which are part of the present invention, are the treatment of (skin) burns, keloid disease or retained placenta, nipple pain and disorders including intervertebral disc herniation, keloid, cellulite, lipoma, Peyronie's disease and Dupuytren's. In view of the improved enzymatic digestion of ECM proteins using an aqueous composition as shown herein, it is also envisaged that an aqueous composition of the invention allows for improved ECM protein breakdown in disorders that are associated with pathological accumulation of extracellular matrix, especially those disorders that are characterized by a pathological accumulation of extracellular matrix in or on skin or in a body orifice or its associated cavity. Preferably, in such treatment methods, an aqueous composition as disclosed herein is topically applied when treatment of skin disorders such as wounds (e.g., non-healing wounds or necrotic wounds), keloid disease, Peyronie's disease and Dupuytren's is envisaged. When treatment of disorders is envisaged wherein pathological accumulation of ECM occurs in a body cavity, other appropriate routes of administration are available to the skilled person, such as by injection in case of pathological accumulation of ECM that occurs in deeper skin layer or intraplacental injection when treating a subject with a retained placenta. Other appropriate routes of administration can be administration through instillation when an aqueous composition as disclosed herein is to be delivered in a body cavity.

### Methods for Treating a Subject With a Cartilage Tissue Injury

In another aspect, a method for enzymatic digestion of the invention is part of a method for treating a subject with a cartilage tissue injury. The term “injury” can be used interchangeably with the term “defect” or “lesion”.

In such a method, during the operative treatment of a subject with a cartilage tissue injury, a method for enzymatic digestion as disclosed herein is performed. In other words, such a method is performed intra-operatively, and may comprise the steps-harvesting or biopsying a cartilage tissue sample, such as meniscus tissue sample, from a subject with a cartilage tissue injury, such as a meniscus injury;—optionally mincing said tissue sample;—performing a method for enzymatic digestion as disclosed herein; isolating chondrocytes and/or chondrons from said aqueous medium after enzymatic digestion; re-seeding, transplanting or implanting said isolated (autologous) chondrocytes and/or chondrons on or in said cartilage tissue of said subject that is injured, preferably at the site where the defect is located. Preferably, the harvested cartilage tissue sample is obtained from the injured cartilage tissue but at a site that is not injured.

In other words, the present invention provides a method for repairing a cartilage defect in a mammal in need thereof comprising the steps of: a) providing a tissue sample of a suitable autologous source of chondrocytes and/or chondrons; b) performing a method for isolating as disclosed herein; c) optionally, mixing the thus isolated chondrocytes and/or chondrons with a matrix gel material; d) optionally, loading said chondrocytes and/or chondrons in or on a scaffold, and e) implanting said chondrocytes and/or chondrons (optionally in said matrix gel) or said loaded scaffold in a cartilage defect.

### NUMBERED EMBODIMENTS

1. A method for enzymatic digestion of an extracellular matrix protein in a tissue sample, comprising the steps of:
providing an aqueous medium that comprises (i) a matrix metalloproteinase and (ii) a cation that is Ca²⁺ at a concentration of at least 2 mmol/L;
contacting said aqueous medium with a tissue sample that comprises an extracellular matrix protein under conditions that allow for enzymatic digestion of said extracellular matrix protein.

2. The method according to embodiment 1, wherein said aqueous medium is prepared by a method comprising the steps of:
providing an aqueous medium;
dissolving a calcium salt in said aqueous medium, wherein the final concentration of Ca²⁺ in said aqueous medium is at least 2 mmol/L; and
mixing, prior or after said step of dissolving, a matrix metalloproteinase with said aqueous medium.

3. The method according to embodiment 1 or embodiment 2, wherein said step of contacting said aqueous medium with said tissue sample provides for at least partial dissociation of said tissue sample thereby releasing a cell from said tissue sample in said aqueous medium.

4. The method according to any one of the preceding embodiments, wherein said method is a method for isolating a cell, either with or without its pericellular matrix, from a tissue sample.

5. The method according to any one of the preceding embodiments, wherein said method further comprises a step of:
isolating a cell from said aqueous medium after enzymatic digestion, preferably by passing a digestate through a cell strainer.

6. The method according to any one of the preceding embodiments, wherein the matrix metalloproteinase is selected from the group consisting of a collagenase, a gelatinase, a stromelysin, a matrilysin, a metalloelastase, an enamelysin, an endometase and an epilysin.

7. The method according to any one of the preceding embodiments, wherein the matrix metalloproteinase is a collagenase.

8. The method according to any one of the preceding embodiments, wherein said aqueous medium comprises dissolved CaCl₂.

9. The method according to any one of the preceding embodiments, wherein said tissue sample is selected from the group consisting of a sample of cartilage tissue, muscle tissue, heart tissue, liver tissue, pancreas tissue, thyroid tissue and salivary gland tissue.

10. The method according to any one of the preceding embodiments, wherein said tissue sample is a harvested or biopsied tissue sample, optionally a harvested or biopsied tissue sample that is minced prior to said step of contacting said aqueous medium with said tissue sample.

11. The method according to any one of embodiments 3-10, wherein said cell is a chondrocyte, either with or without its pericellular matrix, and said tissue sample is a cartilage tissue sample, preferably an articular cartilage tissue sample.

12. An aqueous medium comprising (i) a matrix metalloproteinase and (ii) a cation that is Ca²⁺ at a concentration of at least 2 mmol/L.

13. The aqueous medium according to embodiment 12, wherein said matrix metalloproteinase is a collagenase.

14. The aqueous medium according to embodiment 12 or embodiment 13, wherein said aqueous medium further comprises an articular cartilage tissue sample and/or a chondrocyte.

15. Use of an aqueous medium according to any one of embodiments 12-14 in extracellular matrix degradation; wherein said extracellular matrix comprises an extracellular matrix protein, preferably a collagen.

16. An aqueous medium according to any one of embodiments 12-14 for use as a medicament.

17. The aqueous medium for use according to embodiment 16, wherein said aqueous medium is for use in a method for treating a disorder associated with pathological accumulation of extracellular matrix, preferably pathological accumulation of extracellular matrix in or on skin or in a body orifice or its associated cavity.

18. The aqueous medium for use according to embodiment 16, wherein said aqueous medium is for use in a method of treating a wound, burn, keloid disease or retained placenta, or for use in wound debridement.

19. A method for tenderizing a meat product, comprising the steps of:
providing an aqueous medium that comprises (i) a matrix metalloproteinase and (ii) a cation that is Ca²⁺ at a concentration of at least 2 mmol/L;
contacting said aqueous medium with a meat product that comprises an extracellular matrix protein under conditions that allow for enzymatic digestion of said extracellular matrix protein.

20. A method for deboning a fish product, comprising the steps of:
providing an aqueous medium that comprises (i) a matrix metalloproteinase and (ii) a cation that is Ca²⁺ at a concentration of at least 2 mmol/L;
contacting said aqueous medium with a fish product that comprises an extracellular matrix protein under conditions that allow for enzymatic digestion of said extracellular matrix protein.

21. A method for the production of a peptide or protein hydrolysate, comprising the steps of:
providing an aqueous medium according to any one of embodiments 12-14;
contacting said aqueous medium with a composition comprising a protein under conditions that allow for enzymatic digestion of said protein.

22. The method according to embodiment 21, wherein the peptide is a bioactive peptide such as a collagen peptide and the protein is a collagen.

23. The method according to embodiment 21, wherein the protein hydrolysate is a collagen hydrolysate and the protein is a collagen.

### EXAMPLES

### Example 1

### Isolating Chondrocytes From Articular Cartilage Tissue Samples

### Materials & Methods

Pieces of articular cartilage were harvested from fresh equine metacarpophalangeal or metatarsophalangeal joints and stored in RPMI (Roswell Park Memorial Institute) 1640 medium at room temperature. A 2% collagenase solution was prepared by dissolving 200 mg of lyophilized collagenase powder (Collagenase Type 2, Worthington Biochemical Corporation, Lakewood, New Jersey, USA) in 10 ml of RPMI 1640. The solution was stored at 37° C. during cartilage mincing. The cartilage pieces were minced manually with a scalpel and a known amount of minced cartilage was transferred into a 12-ml tube with 10 ml of the collagenase solution. In the control group, no CaCl₂ was added to this tube. In the experimental group, 96 μl of a 1M CaCl₂ stock solution was added to the tube, resulting in a Ca²⁺ concentration of 10 mM. The tube was incubated on a vortex shaker for 35 minutes at 37° C. The digestate was resuspended gently with a 5-ml pipette and passed through a cell strainer with a pore size of 100 μm into a 50-ml tube. RPMI 1640 was added to the tube to fill it up to 50 ml, after which it was centrifuged for 2 minutes at 750 g. The supernatant was discarded, the cell pellet gently resuspended in 50 ml RPMI 1640, and the tube was centrifuged a second time for 2 minutes at 750 g. The supernatant was again discarded and the cell pellet was gently resuspended in a small amount of 1640 RPMI, from which a 15 μl sample was taken to count living cells using a Bürker-Türk chamber. The outcome was expressed as living cells isolated per gram of articular cartilage.

### Results

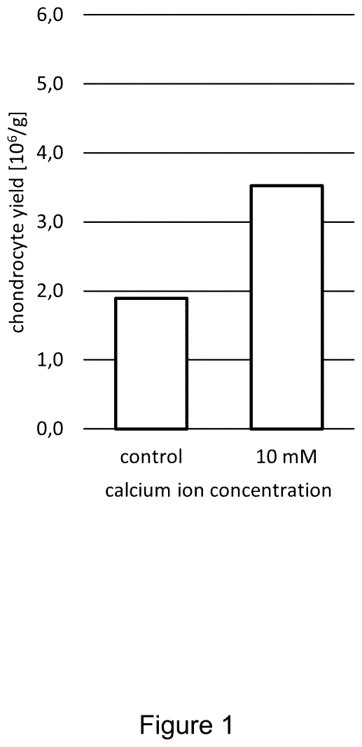

When equine articular cartilage was exposed to a collagenase solution under continuous shaking at 37° C., 1.90 million primary articular chondrocytes were harvested per gram of cartilage without added Ca²⁺. At a concentration of 10 mmol/L of Ca²⁺ in the digestion solution, 3.53 million primary articular chondrocytes were harvested per gram of cartilage; roughly 1.9 times more (FIG. 1).

### Example 2

### Harvesting Chondrocytes From Bovine Articular Cartilage

### Materials & Methods

The materials and methods are as described in Example 1. Pieces of articular cartilage were harvested from fresh bovine metacarpophalangeal or metatarsophalangeal joints. One experimental group was added, resulting in three groups: Control, 3 mM CaCl₂, and 10 mM CaCl₂.

### Results

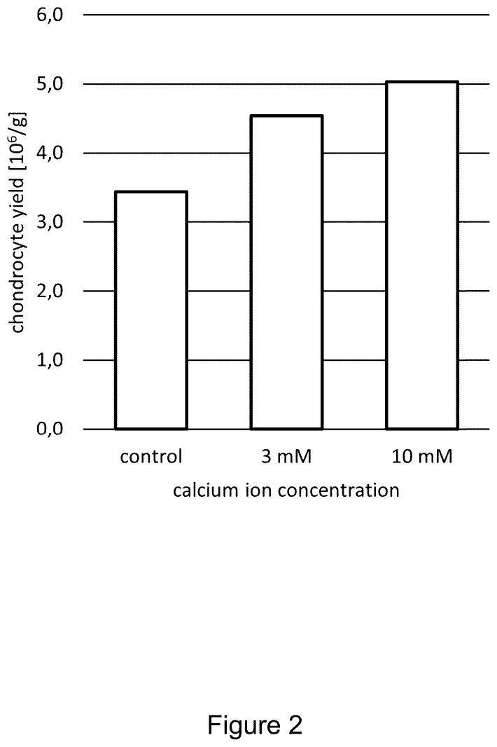

When bovine articular cartilage was exposed to a collagenase solution under continuous shaking at 37° C., 3.44 million primary articular chondrocytes were harvested per gram of cartilage without added Ca²⁺. At a concentration of 3 mmol/L of Ca²⁺ in the digestion solution, 4.54 million primary articular chondrocytes were harvested per gram of cartilage; roughly 1.3 times more than the control group. At a concentration of 10 mmol/L of Ca²⁺ in the digestion solution, 5.03 million primary articular chondrocytes were harvested per gram of cartilage; roughly 1.5 times more than the control group (FIG. 2).

1. A method for enzymatic digestion of an extracellular matrix protein in a tissue sample, comprising the steps of:
providing an aqueous medium that comprises (i) a matrix metalloproteinase and (ii) a cation that is Ca²⁺ at a concentration of at least 2 mmol/L;
contacting said aqueous medium with a tissue sample that comprises an extracellular matrix protein under conditions that allow for enzymatic digestion of said extracellular matrix protein;
wherein said tissue sample is a cartilage tissue sample.

2. The method according to claim 1, wherein said aqueous medium is prepared by a method comprising the steps of:
providing an aqueous medium;
dissolving a calcium salt in said aqueous medium, wherein the final concentration of Ca²⁺ in said aqueous medium is at least 2 mmol/L; and
mixing, prior or after said step of dissolving, a matrix metalloproteinase with said aqueous medium.

3. The method according to claim 1, wherein said step of contacting said aqueous medium with said tissue sample provides for at least partial dissociation of said tissue sample thereby releasing a cell from said tissue sample in said aqueous medium.

4. The method according to claim 1, wherein said method is a method for isolating a cell, either with or without its pericellular matrix, from a tissue sample.

5. The method according to claim 1, wherein said method further comprises a step of:
isolating a cell from said aqueous medium after enzymatic digestion, preferably by passing a digestate through a cell strainer.

6. The method according to claim 1, wherein the matrix metalloproteinase is selected from the group consisting of a collagenase, a gelatinase, a stromelysin, a matrilysin, a metalloelastase, an enamelysin, an endometase and an epilysin.

7. The method according to claim 1, wherein the matrix metalloproteinase is a collagenase, preferably wherein said matrix metalloproteinase is exclusively restricted to a collagenase, more preferably wherein collagenase is the only protease being present in the aqueous medium.

8. The method according to claim 1, wherein said aqueous medium comprises dissolved CaCl₂.

9. The method according to claim 1, wherein said tissue sample is a harvested or biopsied tissue sample, optionally a harvested or biopsied tissue sample that is minced prior to said step of contacting said aqueous medium with said tissue sample.

10. The method according to claim 1, wherein said cell is a chondrocyte, either with or without its pericellular matrix, and preferably said tissue sample is an articular cartilage tissue sample.

11. An aqueous medium comprising (i) a matrix metalloproteinase and (ii) a cation that is Ca²⁺ at a concentration of at least 2 mmol/L.

12. The aqueous medium according to claim 11, wherein said matrix metalloproteinase is a collagenase, preferably wherein said matrix metalloproteinase is exclusively restricted to a collagenase, more preferably wherein collagenase is the only protease being present in the aqueous medium.

13. The aqueous medium according to claim 11, wherein said aqueous medium further comprises an articular cartilage tissue sample and/or a chondrocyte.

14-18. (canceled)

19. A method for tenderizing a meat product, comprising the steps of:
providing an aqueous medium that comprises (i) a matrix metalloproteinase and (ii) a cation that is Ca²⁺ at a concentration of at least 2 mmol/L;
contacting said aqueous medium with a meat product that comprises an extracellular matrix protein under conditions that allow for enzymatic digestion of said extracellular matrix protein.

20. A method for deboning a fish product, comprising the steps of:
providing an aqueous medium that comprises (i) a matrix metalloproteinase and (ii) a cation that is Ca²⁺ at a concentration of at least 2 mmol/L;
contacting said aqueous medium with a fish product that comprises an extracellular matrix protein under conditions that allow for enzymatic digestion of said extracellular matrix protein.

21. A method for the production of a peptide or protein hydrolysate, comprising the steps of:
providing an aqueous medium according to claim 11;
contacting said aqueous medium with a composition comprising a protein under conditions that allow for enzymatic digestion of said protein.

22. The method according to claim 21, wherein the peptide is a bioactive peptide such as a collagen peptide and the protein is a collagen.

23. The method according to claim 21, wherein the protein hydrolysate is a collagen hydrolysate and the protein is a collagen.

24. A method for treating a disorder associated with pathological accumulation of extracellular matrix, said method comprising the step of:
administering a therapeutically effective amount of an aqueous medium according to claim 11 to a subject in need thereof.

25. The method according to claim 24, wherein said disorder is a wound, burn, keloid disease, retained placenta or wound debridement.

26. A method for treating a subject with a cartilage tissue injury, said method comprising the steps of:
harvesting or biopsying a cartilage tissue sample from a subject with a cartilage tissue injury;
optionally mincing said tissue sample;
performing the method for enzymatic digestion of claim 1 on the cartilage tissue sample; isolating chondrocytes and/or chondrons from said aqueous medium after enzymatic digestion; re-seeding or transplanting said isolated (autologous) chondrocytes and/or chondrons on or in said cartilage tissue of said subject that is injured.
